(19) United States
(12) Patent Application Publication
(10) Pub. No.: US 2015/0305426 A1
(43) Pub. Date: Oct. 29, 2015

(54) BICYCLE HELMET WITH INTEGRATED ELECTRONICS

(71) Applicant: Ford Global Technologies, LLC, Dearborn, MI (US)

(72) Inventors: Benjamin Lee, Dearborn, MI (US); Eswar Timmavajjala, Dearborn, MI (US)

(73) Assignee: Ford Global Technologies, LLC, Dearborn, MI (US)

(21) Appl. No.: 14/261,712

(22) Filed: Apr. 25, 2014

(51) Int. Cl. A42B 3/04; A61B 5/00

(52) U.S. Cl. CPC A42B 3/046; A61B 5/0015

(57) ABSTRACT

A bicycle helmet includes a shell, a pad, and a sensor system. The sensor system is at least partially disposed on the shell or the pad. The sensor system can detect a user status and wirelessly transmit a status signal representing the detected user status to a paired device. The paired device can determine whether the user has suffered an injury and whether to contact emergency services. The bicycle helmet may include an integrated microphone and speakers so that the user can speak with an emergency services operator.

### BACKGROUND

Some bicyclists enjoy listening to music while riding. Bicyclists will sometimes wear headphones plugged into a portable music player while riding. Managing the cable between the headphones and the portable music player can be a challenge. Moreover, not all bicycle helmets will accommodate headphones, including in-ear headphones, and not all headphones can effectively reduce wind noise that occurs while bicycling.

### BRIEF DESCRIPTION OF THE DRAWINGS

FIG. 1 illustrates an exemplary system with a bicycle helmet having integrated electronics for communicating with a mobile device, a bicycle, or both.

FIG. 2 is a block diagram illustrating exemplary components of the bicycle helmet.

FIG. 3 illustrates an exemplary holder for securing the mobile device to the bicycle.

FIG. 4 is a flowchart of an exemplary process that may be used by the mobile device during communication with the bicycle helmet.

### DETAILED DESCRIPTION

An exemplary bicycle helmet includes a shell, a pad, and a sensor system. The sensor system is at least partially disposed on the shell or the pad. The sensor system can detect a user status and wirelessly transmit a status signal representing the detected user status to a paired device such as a mobile device. With the status signal, the mobile device can determine whether the user has suffered an injury and whether to contact emergency services. The bicycle helmet may include an integrated microphone and speakers so that the user can speak with an emergency services operator. Furthermore, the mobile device may wirelessly stream music or other audio to the speakers of the bicycle helmet. In some possible implementations, the bicycle helmet may pair with and collect data from the bicycle itself.

The elements shown in the FIGS. may take many different forms and include multiple and/or alternate components and facilities. The exemplary components illustrated are not intended to be limiting. Indeed, additional or alternative components and/or implementations may be used. Moreover, the components illustrated are not necessarily drawn to scale.

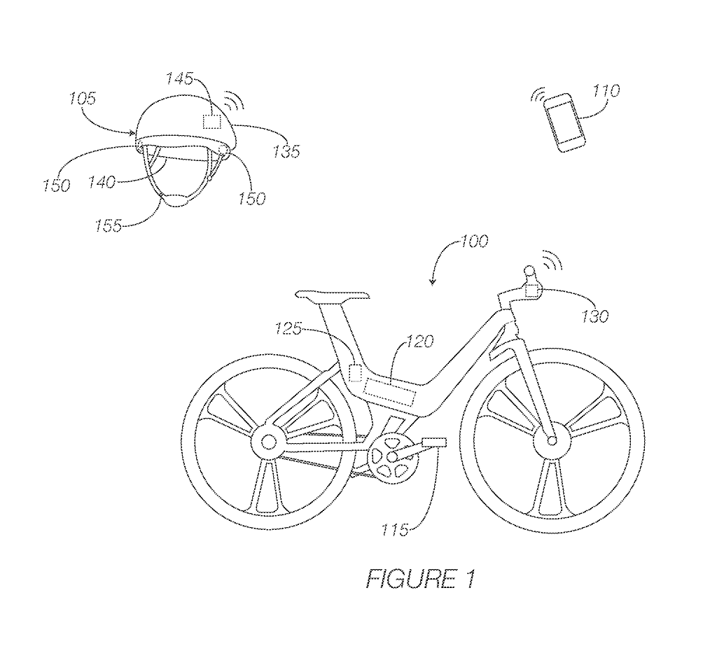

As illustrated in FIG. 1, a bicycle 100, a bicycle helmet 105 to be worn by a user, and a mobile device 110 are shown. The bicycle 100 may include any human or electrically powered bicycle 100. For instance, the bicycle 100 may include pedals 115 that, when pushed, rotate, e.g., a gear and chain assembly to propel the vehicle forward. Alternatively or in addition, the bicycle 100 may include a motor 120 configured to propel the vehicle in accordance with a current supplied by a battery 125. The bicycle 100 may be configured to pair with the bicycle helmet 105, the mobile device 110, or both. For instance, the bicycle 100 may include a communication module 130 configured to transmit signals to, and receive signals from, the bicycle helmet 105 and the mobile device 110. The communication module 130 may be configured to transmit information about the bicycle 100. The information may include, e.g., the state of charge of the battery 125, a system status update, a distance traveled, a number of rotations of the wheels, a speed, or the like.

The bicycle helmet 105 may be worn by a user during operation of the bicycle 100. The bicycle helmet 105 may include a shell 135 disposed on a pad 140. The shell 135 may be formed from plastic or another rigid material. The pad 140 may be formed from foam, although other materials may be used. In addition, the bicycle helmet 105 may include a sensor system 145 having components disposed on the pad 140, the shell 135, or both. The sensor system 145 may be configured to detect, using various sensors discussed in greater detail below, a user status. Moreover, the sensor system 145 may be configured to transmit a status signal representing the detected user status to, e.g., the mobile device 110 or any other device paired with the bicycle helmet 105, as discussed in greater detail below. Examples of user statuses transmitted by the sensor system 145 may include a change in acceleration, which may indicate that the user has fallen off the bicycle 100 or been involved in an accident, or a physiological parameter that may indicate that the user is having a medical emergency such as a heart attack.

The sensor system 145 may transmit the user status to, e.g., the mobile device 110. The sensor system 145 may, therefore, be configured for wired communication, wireless communication, or both. Examples of wireless communication may include communication in accordance with the Bluetooth® protocol. The bicycle helmet 105 may further include speakers 150 and a microphone 155. The speakers 150 may be at least partially embedded in the pad 140, and the microphone 155 may be disposed on a chin strap or another area near a user's mouth. The speakers 150 and microphone 155 may be wired to the sensor system 145, and the sensor system 145 may be configured to control the operation of the speakers 150 and microphone 155. For instance, the sensor system 145 may be configured to enable or disable the speakers 150, microphone 155, or both at various times and under various circumstances, discussed in greater detail below.

The mobile device 110 may include a wireless communication device such as a cellular phone. In some possible implementations, the mobile device 110 may be configured to pair with the bicycle 100, the bicycle helmet 105, or both according to any number of communication protocols, such as Bluetooth®. The mobile device 110 may receive, from the sensor system 145, the status signal representing the detected user status, process the status signal, and determine whether to contact emergency services based on the status signal. In one possible approach, the mobile device 110 may include a navigation system configured to determine the geographic location of the mobile device 110. For instance, the mobile device 110 may include a Global Positioning System (GPS) receiver configured to triangulate the position of the mobile device 110 relative to satellites or terrestrial based transmitter towers. The navigation system, therefore, may be configured for wireless communication. In some possible approaches, the mobile device 110 may include a user interface device such as a touch-sensitive display screen. Moreover, the mobile device 110 may be configured to execute one or more applications. The rider may interact with the mobile device 110 by providing inputs to the user interface device, and the user inputs may be acted on by a processor in accordance with the running application. Moreover, the mobile device 110 may present information to the rider via the user interface device. Examples of information provided to the user may include information about the bicycle 100 such as battery state-of-charge, a system status update, a distance traveled, a number of rotations of the wheels, a speed of the bicycle 100, etc. Alternatively or in addition, a map generated by the navigation system may be presented to the rider via the user interface device.

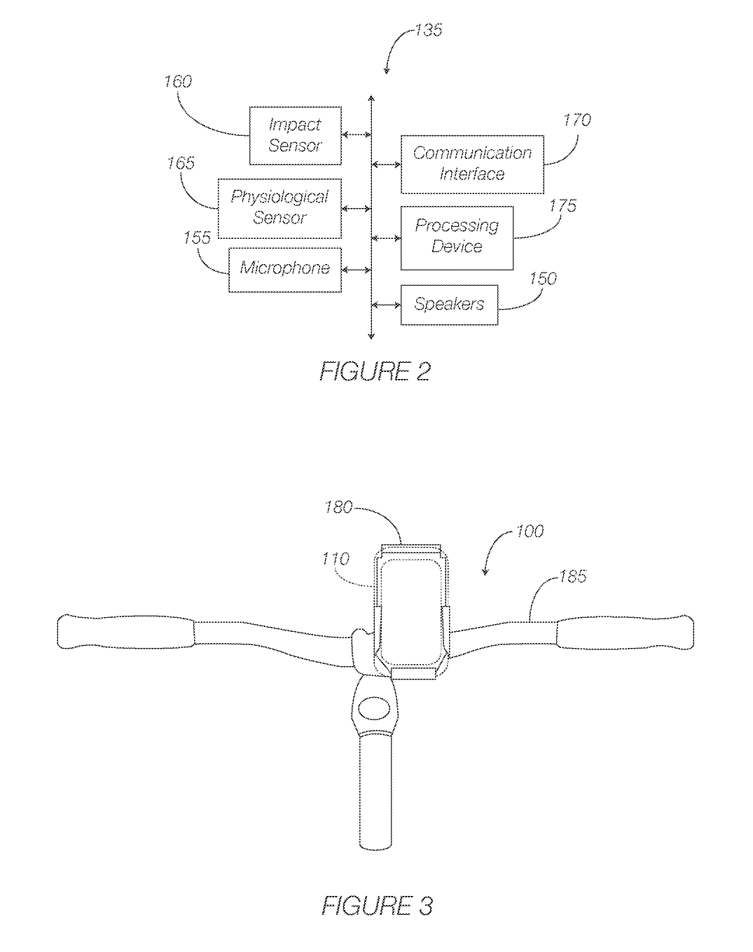

Referring now to FIG. 2, the sensor system 145 incorporated into the bicycle helmet 105 may include an impact sensor 160, a physiological sensor 165, a communication interface 170, and a processing device 175.

The impact sensor 160 may be configured to detect a change in acceleration and generate a status signal in accordance with the detected change in acceleration. Therefore, the impact sensor 160 may include an accelerometer. A change in acceleration may indicate a sudden force applied to the bicycle helmet 105, which may occur if the rider falls off the bicycle 100 or otherwise hits his or her head on an object, such as a tree branch. If the change in acceleration exceeds a predetermined threshold, the status signal may indicate that the rider has suffered a potentially serious injury.

The physiological sensor 165 may be configured to measure a physiological parameter of the rider and generate a status signal in accordance with the physiological parameter measured. Examples of physiological parameters may include the rider's heart rate, oxygen saturation, or the like. Thus, the physiological sensor 165 may include a heart rate monitor, an oximeter, or any other device capable of making such physiological measurements. In one possible implementation, the physiological sensor 165 may include a light source and a photodetector. The physiological sensor 165 may be configured to measure physiological parameters based on, e.g., the amount of light scattering, reflections, or both caused by tissue or blood between the light source and the light detector.

The communication interface 170 may be configured to facilitate pairing and wireless communication with the bicycle 100, the mobile device 110, or both according to any number of wireless communication protocols. For instance, the communication interface 170 may be configured to communicate in accordance with the Bluetooth® protocol. In one possible approach, the communication interface 170 may be configured to receive status signals generated by the impact sensor 160, the physiological sensor 165, or both, and transmit signals to any device paired with the sensor system 145 such as the bicycle 100 or the mobile device 110. Furthermore, the communication interface 170 may be configured to receive signals from any paired device, such as the bicycle 100 or mobile device 110. For example, audio signals transmitted from the mobile device 110 to the sensor system 145 may be received via the communication interface 170, processed via the processing device 175, and relayed to the speakers 150. In addition, signals generated by the microphone 155 may be transmitted to the mobile device 110 via the communication interface 170. Therefore, the speakers 150, the microphone 155, or both may receive signals from the communication interface 170.

The processing device 175 may be configured to process signals received from the impact sensor 160, the physiological sensor 165, and the communication interface 170. For instance, the processing device 175 may be configured to process the status signal and any signals generated by the microphone 155 into a form readable by the mobile device 110 and command the communication interface 170 to transmit such signals to the mobile device 110. Moreover, the processing device 175 may be configured to process signals received from the bicycle 100 and the mobile device 110 so that such signals may be received and acted upon by the speakers 150, the impact sensor 160, the physiological sensor 165, or the communication interface 170.

In some possible implementations, the processing device 175 may be configured to receive location information from the mobile device 110. With the location information, the processing device 175 may be configured to apply a location-dependent setting for the bicycle helmet 105. The location-dependent settings may consider laws concerning the use of headphones while operating a bicycle 100. Examples of location-dependent settings may include disabling one or more speakers 150. If only one speaker is disabled, another location-dependent setting may include changing an audio output from stereo to mono.

FIG. 3 illustrates an exemplary holder 180 for securing the mobile device 110 to the bicycle 100. As shown, the holder 180 may be attached to handlebars 185 used to steer the bicycle 100. The holder 180 may be configured to secure the mobile device 110 in a location and orientation that allows the mobile device 110 to be seen by the rider during operation of the bicycle 100. In some possible approaches, the holder 180 may include a port for interfacing with the mobile device 110 and allowing the bicycle 100 and the mobile device 110 to engage in wired communication.

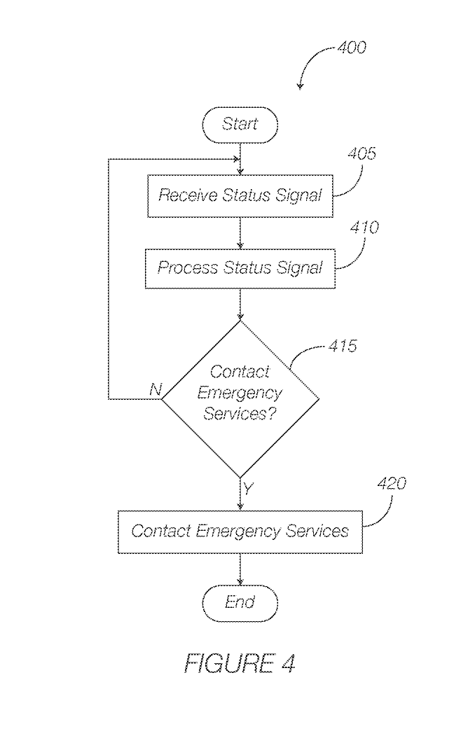

FIG. 4 is a flowchart of an exemplary process 400 that may be used by the mobile device 110 during communication with the bicycle helmet 105. For instance, the process 400 may be executed in accordance with an application installed on the mobile device 110.

At block 405, the mobile device 110 may receive a status signal. The status signal, as discussed above, may represent a user status as measured by the sensor system 145. The sensor system 145 may include an impact sensor 160 that measures a change in acceleration, a physiological sensor 165 that measures a physiological parameter such as heart rate or oxygen saturation, or both. The sensor system 145 may be incorporated into the bicycle helmet 105, and the status signal may be received wirelessly by the mobile device 110.

At block 410, the mobile device 110 may process the status signal. As discussed above, the mobile device 110 may include a processor for processing signals received from the bicycle 100 or the bicycle helmet 105. In some instances, however, some or all of the processing of the status signal may be performed by the processing device 175 of the sensor system 145 prior to the transmission of the status signal to the mobile device 110.

At decision block 415, the mobile device 110 may determine whether to contact emergency services. For instance, the mobile device 110 may determine whether the user status indicates that the rider has been involved in an accident or has suffered a medical emergency. If so, the process 400 may continue at block 420. If not, the process 400 may return to block 405 to await additional status signals.

At block 420, the mobile device 110 may contact emergency services. Contacting emergency services may include calling an emergency number such as 911, enabling the speakers 150 of the bicycle helmet 105, and enabling the microphone 155 of the bicycle helmet 105. In some instances, the rider may not be able to speak with or hear emergency services personnel during the call. Therefore, contacting emergency services may include sending a text-based message from the mobile device 110 using a protocol such as the Short Messaging Service (SMS) protocol. The process 400 may end after block 420.

In general, the computing systems and/or devices described above may employ any of a number of computer operating systems, including, but by no means limited to, versions and/or varieties of the Ford Sync® operating system, the Microsoft Windows® operating system, the Unix operating system (e.g., the Solaris® operating system distributed by Oracle Corporation of Redwood Shores, Calif.), the AIX UNIX operating system distributed by International Business Machines of Armonk, N.Y., the Linux operating system, the Mac OS X and iOS operating systems distributed by Apple Inc. of Cupertino, Calif., the BlackBerry OS distributed by Research In Motion of Waterloo, Canada, and the Android operating system developed by the Open Handset Alliance. Examples of computing devices include, without limitation, an on-board vehicle computer, a computer workstation, a server, a desktop, notebook, laptop, or handheld computer, or some other computing system and/or device.

Computing devices generally include computer-executable instructions, where the instructions may be executable by one or more computing devices such as those listed above. Computer-executable instructions may be compiled or interpreted from computer programs created using a variety of programming languages and/or technologies, including, without limitation, and either alone or in combination, Java™, C, C++, Visual Basic, Java Script, Perl, etc. In general, a processor (e.g., a microprocessor) receives instructions, e.g., from a memory, a computer-readable medium, etc., and executes these instructions, thereby performing one or more processes, including one or more of the processes described herein. Such instructions and other data may be stored and transmitted using a variety of computer-readable media.

A computer-readable medium (also referred to as a processor-readable medium) includes any non-transitory (e.g., tangible) medium that participates in providing data (e.g., instructions) that may be read by a computer (e.g., by a processor of a computer). Such a medium may take many forms, including, but not limited to, non-volatile media and volatile media. Non-volatile media may include, for example, optical or magnetic disks and other persistent memory. Volatile media may include, for example, dynamic random access memory (DRAM), which typically constitutes a main memory. Such instructions may be transmitted by one or more transmission media, including coaxial cables, copper wire and fiber optics, including the wires that comprise a system bus coupled to a processor of a computer. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge, or any other medium from which a computer can read.

Databases, data repositories or other data stores described herein may include various kinds of mechanisms for storing, accessing, and retrieving various kinds of data, including a hierarchical database, a set of files in a file system, an application database in a proprietary format, a relational database management system (RDBMS), etc. Each such data store is generally included within a computing device employing a computer operating system such as one of those mentioned above, and are accessed via a network in any one or more of a variety of manners. A file system may be accessible from a computer operating system, and may include files stored in various formats. An RDBMS generally employs the Structured Query Language (SQL) in addition to a language for creating, storing, editing, and executing stored procedures, such as the PL/SQL language mentioned above.

In some examples, system elements may be implemented as computer-readable instructions (e.g., software) on one or more computing devices (e.g., servers, personal computers, etc.), stored on computer readable media associated therewith (e.g., disks, memories, etc.). A computer program product may comprise such instructions stored on computer readable media for carrying out the functions described herein.

With regard to the processes, systems, methods, heuristics, etc. described herein, it should be understood that, although the steps of such processes, etc. have been described as occurring according to a certain ordered sequence, such processes could be practiced with the described steps performed in an order other than the order described herein. It further should be understood that certain steps could be performed simultaneously, that other steps could be added, or that certain steps described herein could be omitted. In other words, the descriptions of processes herein are provided for the purpose of illustrating certain embodiments, and should in no way be construed so as to limit the claims.

Accordingly, it is to be understood that the above description is intended to be illustrative and not restrictive. Many embodiments and applications other than the examples provided would be apparent upon reading the above description. The scope should be determined, not with reference to the above description, but should instead be determined with reference to the appended claims, along with the full scope of equivalents to which such claims are entitled. It is anticipated and intended that future developments will occur in the technologies discussed herein, and that the disclosed systems and methods will be incorporated into such future embodiments. In sum, it should be understood that the application is capable of modification and variation.

All terms used in the claims are intended to be given their broadest reasonable constructions and their ordinary meanings as understood by those knowledgeable in the technologies described herein unless an explicit indication to the contrary is made herein. In particular, use of the singular articles such as “a,” “the,” “said,” etc. should be read to recite one or more of the indicated elements unless a claim recites an explicit limitation to the contrary.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

1. A bicycle helmet comprising:
a shell;
a pad disposed on the shell;
a sensor system at least partially disposed on at least one of the shell and the pad, wherein the sensor system is configured to detect a user status and transmit a status signal representing the detected user status to a paired device.

2. The bicycle helmet of claim 1, wherein the sensor system includes an impact sensor configured to detect a change in acceleration and generate the status signal in accordance with the change in acceleration.

3. The bicycle helmet of claim 1, wherein the sensor system includes a physiological sensor configured to measure a physiological parameter and generate the status signal in accordance with the measured physiological parameter.

4. The bicycle helmet of claim 3, wherein the physiological parameter includes at least one of heart rate and oxygen saturation.

5. The bicycle helmet of claim 1, wherein the sensor system includes a communication interface is configured to transmit signals to and receive signals from the paired device in accordance with a communication protocol.

6. The bicycle helmet of claim 5, further comprising:
speakers at least partially embedded in the pad and in communication with the communication interface; and
a microphone in communication with the communication interface.

7. The bicycle helmet of claim 6, wherein the communication interface is configured to relay signals from the paired device to the speakers.

8. The bicycle helmet of claim 6, wherein the communication interface is configured to transmit signals from the microphone to the paired device.

9. A method comprising:
receiving, from a sensor system incorporated into a bicycle helmet, a status signal representing a user status measured by the sensor system;
processing, via a computing device, the status signal; and
determining whether to contact emergency services based at least in part on the status signal.

10. The method of claim 9, wherein the sensor system includes an impact sensor, and wherein the status signal represents a change in acceleration measured by the impact sensor.

11. The method of claim 9, wherein the sensor system includes a physiological sensor, and wherein the status signal represents a physiological parameter measured by the physiological sensor.

12. The method of claim 11, wherein the physiological parameter includes at least one of heart rate and oxygen saturation.

13. A system comprising:
a mobile device;
a bicycle helmet having a sensor system configured to pair with the mobile device, wherein the sensor system is further configured to detect a user status and transmit a status signal representing the detected user status to the mobile device.

14. The system of claim 13, wherein the sensor system includes an impact sensor configured to detect a change in acceleration and generate the status signal in accordance with the change in acceleration.

15. The system of claim 13, wherein the sensor system includes a physiological sensor configured to measure a physiological parameter and generate the status signal in accordance with the measured physiological parameter.

16. The system of claim 15, wherein the physiological parameter includes at least one of heart rate and oxygen saturation.

17. The system of claim 13, wherein the sensor system includes a communication interface is configured to transmit signals to and receive signals from the mobile device in accordance with a communication protocol.

18. The system of claim 17, further comprising:
speakers at least partially embedded in the bicycle helmet and in communication with the communication interface; and
a microphone in communication with the communication interface.

19. The system of claim 18, wherein the communication interface is configured to relay signals from the mobile device to the speakers.

20. The system of claim 18, wherein the communication interface is configured to transmit signals from the microphone to the mobile device.
